(19) United States
(12) Patent Application Publication
(10) Pub. No.: US 2011/0268688 A1
(43) Pub. Date: Nov. 3, 2011

(54) METHOD AND COMPOSITIONS FOR IMPROVING SKIN AND BODY APPEARANCE

(76) Inventors: James Timothy McCarthy, Babylon, NY (US); Edward Pelle, Valley Stream, NY (US); Daniel H. Maes, Huntington, NY (US); Nadine A. Pernodet, Huntington Station, NY (US)

(21) Appl. No.: 12/993,549

(22) PCT Filed: Jun. 17, 2009
(86) PCT No.: PCT/US09/47620

§ 371 Date: Mar. 1, 2011

Related U.S. Application Data
(62) Division of application No. 61/075,530, filed on Jun. 25, 2008.

(51) Int. Cl. A61K 8/97; A61Q 19/06; A61Q 19/00

(52) U.S. Cl. 424/78.03

(57) ABSTRACT

A topical composition comprising at least one adipogenesis inhibitor and at least one adipocyte lipolytic agent and, optionally, at least one skin tightening agent; a method for improving the appearance of facial or body skin, treating the appearance of cellulite, or treating the face or body to improve the appearance of skin and uneven skin surfaces.

### TECHNICAL FIELD

The invention is in the field of methods and compositions for treating the skin and body to improve the appearance of skin imperfections such as dimpled skin, cellulite, uneven skin surfaces, fatty deposits and the like or to sculpt facial or body surfaces.

### BACKGROUND OF THE INVENTION

As women age they often develop a condition referred to as cellulite, especially on the backs of legs, pelvic region, or abdomen. The cause of cellulite is very poorly understood. It is most often found in females, rarely in males. Some believe that it is due to the inflexible nature of female connective tissue such that when a woman gains weight and her fat cells expand they push against the inflexible connective tissue and form dimpled skin, which is also commonly referred to as cottage cheese skin. There are no known effective treatments for cellulite. Some propose that rigorous diet and exercise. Cosmetics companies offer various types of creams that are alleged to have cellulite reducing properties when used over time. However, there are problems with all of these approaches and the commercial products that currently exist contain ingredients that treat the symptoms rather than the cause.

Undesired fatty deposits are also found beneath the eyes—commonly referred to as eye bags; beneath the upper arms, under the chin, and in certain areas of the face. In many cases consumers feel their cheeks are “too fat” and they want the sculpted, high-cheekbone look of high fashion models.

Scientific research has shown that subcutaneous adipose tissue contains cell populations that are capable of differentiation into adipocytes. Mature adipocytes store fat, usually in the form of triglycerides. Adipocytes are also capable of increasing in size as more and more fat is stored. For example, weight gain may be a combination of adipogenesis and increased fat storage in existing adipocytes. In the case where an individual loses weight the fat stores in the adipocytes are decreased and the cells become smaller.

Specifically, fat cells, or adipocytes, arise from preadipocytes, which in turn are produced by certain types of stem cells. The process is triggered by accumulation of triglycerides in the body, sometimes due to excessive food intake. The fats are stored in currently existing adipocytes. If the amount of triglycerides becomes too large for the storage capacity of the currently existing adipocytes, the genetic signaling pathway of precursor cells is triggered, causing them to begin to store small triglyceride globules. The change from non-adipocyte or pre-adipocyte to adipocyte occurs in a number of steps. Mature adipocytes tend to be very long-lived and are relatively resistant to apoptosis.

Thus, in order to have the maximum effect on improving the appearance of cellulite, uneven skin surfaces, or the appearance of fatty deposits on the body or face it is desirable to consider both adipogenesis and the increased fat stores found in existing adipocytes. Thus topically applied compositions that contain ingredients that have adipogenesis inhibition activity as well as lipolytic activity—that is the ability to reduce the fat stores found in existing adipocytes, are likely to provide the most efficacious products.

Accordingly, it has been discovered that topically applied compositions comprising at least one adipogenesis inhibitor and at least one adipocyte lipolytic agent provide and, optionally, at least one skin tightening agent, provide improved effects in treating facial and body skin for improvement. The combination of ingredients will improve the appearance of cellulite, uneven skin surfaces, and otherwise promote sculpting of facial and body contours.

It is an object of the invention to provide a topical composition for treating cellulite or sculpting facial or body contours.

It is a further object of the invention to provide a topical composition and method for improving the appearance of perceived fatty deposits on facial or bodily surfaces, such as beneath the eyes, under the arms, neck, or in certain areas of the face by applying the composition of the invention.

It is a further object of the invention to provide a method for treating the body to improve the appearance of dimpled skin or uneven skin surfaces by applying a composition containing at least on adipogenesis inhibitor and at least one adipocyte lipolytic agent and, optionally, at least one skin tightening agent.

It is a further object of the invention to provide a method for improving the appearance of cellulite by topically applying a composition containing at least one adipogenesis inhibitor and at least one adipocyte lipolytic agent and, optionally, at least one skin tightening agent.

It is a further object of the invention to provide a method for sculpting facial or body surfaces by topically applying a composition containing at least one adipogenesis inhibitor and at least one adipocyte lipolytic agent and, optionally, at least one skin tightening agent.

It is a further object of the invention to provide a method and compositions for improving the appearance of the body by topically applying a composition containing at least one adipogenesis inhibitor and at least one adipocyte lipolytic agent and, optionally, at least one skin tightening agent.

### SUMMARY OF THE INVENTION

The invention is directed to a topical composition comprising at least one adipogenesis inhibitor and at least one adipocyte lipolytic agent.

The invention is also directed to a method for treating cellulite by topically applying a composition comprising at least one adipogenesis inhibitor and at least one adipocyte lipolytic agent.

The invention is further directed to a method for sculpting facial or bodily surfaces by applying a composition comprising at least one adipogenesis inhibitor and at least one adipocyte lipolytic agent.

The invention is further directed to a method for improving the appearance of skin by applying a composition comprising at least one adipogenesis inhibitor and at least one adipocyte lipolytic agent.

### DESCRIPTION OF THE FIGURES

FIG. 1 shows the tabulated measurement of adipocyte lipolytic activity of forskolin and conjugated linoleic acid.

FIG. 2 shows the tabulated measurements of adipocyte differentiation inhibition properties of resveratrol, resveratrate, resveratryl ferulate and Longevicell™.

### DETAILED DESCRIPTION

### Definitions

“Adipocyte” means a connective tissue cell that has differentiated and become specialized in the synthesis and storage of fat.

“Adipogenesis inhibitor” means an ingredient that is capable of inhibiting the differentiation of connective tissue cells into adipocytes, or inhibiting the any one or more steps of the adipogenesis pathway. By way of example, the adipogenesis inhibitor may completely inhibit the differentiation of a pre-adipocyte into an adipocyte, or it may inhibit a cell that has already begun the adipogenesis process but is still in one or more of the non-final stages leading up to the formation of a mature adipocyte.

“Adipocyte lipolytic agent” means an ingredient that is capable of causing a reduction in the fat stores of adipocytes. “Cellulite” means the dimpled, bulged, or rippled appearance associated with subcutaneous fat on the hips, thighs, or buttocks.

“Sculpting” means with respect to facial or body skin, the ability to improve the appearance of that skin, including improving appearance of fat deposits on facial or body skin by causing them to appear less prominent or noticeable. “Skin Tightening Agent” means an ingredient that, when applied to skin, will cause the skin to appear more taut and/or improve the laxity of the skin.

### The Compositions of the Invention

The compositions of the invention are topical and comprise at least one adipogenesis inhibitor and at least one adipocyte lipolytic agent, which may be found in a suitable topical carrier comprising one or more ingredients.

The Adipogenesis Inhibitor

The composition of the invention comprises at least one adipogenesis inhibitor. It should be present in the composition an amount sufficient to exert some inhibitory effect on the generation of adipocytes in the area to which the composition is applied. Generally, suitable ranges of adipogenesis inhibitor may be from about 0.001 to 60%, preferably from about 0.05 to 55%, preferably from about 0.1 to 50% by weight of the total composition. In general, suitable adipogenesis inhibitors may be determined by conducting a simple test using an adipogenesis assay test kit purchased from Zen-Bio, www.zen-bio.com. One preferred kit is the Adipogenesis Assay Kit, PPARγ agonist (reagents+cells) having catalog no. DIF-AG purchased from Zen-Bio.

Suitable adipogenesis inhibitors include, but are not limited to, resveratrol, esters of resveratrol and inorganic acids such as phosphoric, sulfuric, hydrochloric acids and the like; esters of resveratrol and carboxylic acids such as ferulic acid, hydrolyzed Myrtus Communis leaf extract (sold under the trademark Longevicell® by Silab), and the like. Particularly suitable are trisodium resveratrol triphosphate, resveratrol, resveratrol ferulate, and Myrtus Communis leaf extract. A variety of other ingredients are also known to have adipocyte differentiation activity, including but not limited to COX-2 (cyclooxygenase-2) inhibitors (such as extracts of plants from the Acacia genus, Calluna Vulgaris, Rosa Canina fruit extract, nettle leaf extract, Polyporus Umbellatus, Chamomilla Recutita (Matricaria) flower oil, Coleus Forskohlii extract, Rusperin C® (a mixture of Butcher's Broom extract and heperidin methyl chalcone), HOP's beta acids, and mixtures thereof); TRB3 or Tribble 3, a kinase; calcineurin; C75, a fatty acid synthase inhibitor; tripeptides of the sequence Val-Tyr-Pro or Val-Thr-Leu; A-type lamnins; various types of calpain inhibitors; mifepristone; protein kinase C activators; lipooxygenase inhibitors such as Haplophyllum hispanicum Spach, Scutellaria Rivularis extracts and its active components, alpha ketoboswellic acid, Zileuton® (1-(1-benzothiophen-2-ylethyl)-1-hydroxy-urea), Centaurium extract, nordihydroguaiaretic acid, ginger oil, resveratrol, retinol, Polygonum Cuspidatum extract, cedar himalaya extract, tetrahydrocurcuminoids, Nigella Sativa essential oil, copaiba balsam, Australian sandalwood, Rhodiola Rosea, frankincense, blue cypress oil, blue chamomile oil, anti-inflammatory oil blend, vetiver mada, bulgarian rose oil, resveratrol ferulate, Helichrysum oil, rosmarinic acid, resveratrol triphosphate, noni leaf extract; stromelysin-3; endrin; glucocorticoids; Caralluma Fimbriata Extract, Mucuna Extract, Bacana Extract, G-alpha 13 inhibitors such as those disclosed WO/2003/10449, hereby incorporated by references in its entirety. Such G-alpha 13 inhibitors include antibodies, peptides (including dominant negative peptides) and antisense compounds, including ribozymes, inhibitory RNA molecules including siRNA molecules and antisense oligonucleotides; and so on.

The Adipocyte Lipolytic Agent

The compositions of the invention also comprise at least one adipocyte lipolytic agent. Preferably it is present in an amount sufficient to cause the adipocytes in the area to which the composition is applied to reduce their fat stores. Suggested ranges of the adipocyte lipolytic agent are from about 0.01 to 50% preferably from about 0.05 to 45%, more preferably from about 0.1 to 40% by weight of the total composition.

Suitable lipolytic agents may be identified by conducting a simple test using a lipolysis assay kit purchased from Zen-Bio. One suitable kit is the Lypolysis Assay Kit (dual glycerol and free fatty acids detection, reagents+cells) purchased from Zen-Bio (www.zen-bio.com) under catalog no. LIP-3.

Suitable lipolytic agents include conjugated linoleic acid; forskolin. Other ingredients are known to be lipolytic agents as well, such as nicotine; deoxycholate; theophylline; caffeine; theobromine; magnolol (a fraction of Magnolia extract); isoproterenol; kaempferol-7-O-Neohesperidoside; Clary extract; Chrystanthellum extract, specifically Chrysanthellum Indicum; and the like.

### The Skin Tightening Agent

The composition optionally contains at least one skin tightening agent. In certain preferred embodiments, this agent is present. Suggested ranges are from about 0.01 to 70%, preferably from about 0.05 to 65%, more preferably from about 0.1 to 60% by weight of the total composition. Suitable skin tightening agents include ingredients that have a contractile effect when applied to the skin, such as silicone elastomers, various polymers or copolymers, and the like.

Polymeric or copolymeric ingredients include homo- or copolymers of acrylates, methacrylates, acrylamides, methacrylamides, urethanes, and the like, which may be copolymerized or derivatized with long chain alkyl, ether, or alkoxy groups. Examples include acrylamides copolymer, acrylamide/sodium acrylate copolymer, ammonium acryloyldimethyl taurate, ammonium acryloyldimethyltaurate/VP copolymer, acrylates C10-30 alkyl acrylate crosspolymer, acrylates/beheneth-25 methacrylate copolymer, acrylates/vinyl neodecanoate copolymer, acrylates ceteth-20 itaconate copolymer, acrylates ceteth-20 methacrylate copolymer, acrylates steareth-20 methacrylate copolymer, acrylates steareth-50 methacrylate copolymer, ammonium acryloyldimethyltaurate/steareth-8 methacrylate copolymer, acrylates/palmeth-25 itaconate copolymer, acrylates/palmeth-25 acrylate copolymer, and the like.

Also suitable as as skin tightening agents are various types of silicone elastomers, that is crosslinked silicones formed by the reaction of vinyl-terminated siloxanes and methylhydroxydimethyl siloxanes. Examples of such silicone elastomers include those sold under the trademarks Gransil™ by Grant Industries, Botanisil™ by Botanigenics, Actiprime™ by Active Organics, KSG or USG by Shin-Etsu Silicones; or 9040, 9044 silicone elastomers or blends thereof by Dow Corning Corporation. Such elastomers include but are not limited to cetearyl dimethicone/vinyl dimethicone crosspolymer, dimethicone/vinyl dimethicone crosspolymer, stearyl dimethicone/vinyl dimethicone crosspolymer, cetyl dimethicone bis-vinyl dimethicone crosspolymer, dimethicone crosspolymer-3, dimethicone/divinyl dimethicone/silsesquioxane crosspolymer, diphenyl dimethicone/vinyl dimethicone crosspolymer, polydimethylsiloxyethyldimethicone/bis-vinyl dimethicone crosspolymer, and so on.

### Other Ingredients

A variety of other ingredients may be incorporated into the composition to provide a cosmetically acceptable vehicle for the adipogenesis inhibitor and the adipocyte lipolytic agent. The cosmetically acceptable vehicle may be in the form of an emulsion—that is water in oil or oil in water emulsion. Alternatively, the composition may be an aqueous suspension, gel, or solution; or it may be anhydrous. The composition may be in the liquid, solid, or semi-solid form. If water is present, the composition may comprise from about 0.1 to 99%, preferably from about 0.5 to 95%, more preferably from about 1 to 90% by weight of the total composition of water. In the event the composition is in the form of an emulsion, in addition to the water, the composition may comprise from about 0.1 to 90%, preferably from about 0.5 to 85%, more preferably from about 1 to 80% by weight of the total composition of oil. The composition may additionally contain one or more of humectants, thickening or structuring agents, surfactants, botanicals, preservatives, oils, and so on.

Oils

Suitable oils may be volatile or non-volatile, silicone, or organic. The oils are preferably pourable at room temperature.

The term “volatile” means that the oil has a vapor pressure of at least about 2 mm of mercury at 20° C. The term “nonvolatile” means that the oil has a vapor pressure of less than about 1 mm of mercury at 20° C. Suitable volatile silicone oils include linear, branched, or cyclic silicones such as hexamethyldisiloxane, having a viscosity of about 0.65 centistokes at 25° C.; octamethyltrisiloxane, having a viscosity of about 1.0 centistokes at 25° C.; decamethyltetrasiloxane, having a viscosity of about 1.5 centistokes at 25° C.; dodecamethylpentasiloxane, having a viscosity of about 2.0 centistokes at 25° C.; octamethylcyclotetrasiloxane; decamethylcyclopentasiloxane; dodecamethylcyclohexasiloxane; or a branched volatile silicone, methyl trimethicone having a viscosity of about 1.5 centistokes at 25° C.

Also suitable are volatile hydrocarbon oils including paraffinic hydrocarbons such as isododecane, isohexadecane or mixtures thereof.

Nonvolatile oils may be silicone or organic oils. Examples of suitable nonvolatile silicone oils are dimethicone; lower alkyl chain dimethicones such as cetyl dimethicone; phenyl trimethicone; phenyl dimethicone; trimethylsiloxyphenyl dimethicone; amodimethicone; cetearyl dimethicone; dimethiconol; lauryl dimethicone; phenethyl dimethicone; diethysiloxane; and so on.

Suitable organic oils include esters and hydrocarbons. Suitable esters may be mono-, di-, or triesters, that is C1-40 carboxylic acid esters of C1-40 aliphatic or aromatic alcohols where the acid may contain one or more carboxylic acid groups or the alcohol may contain one or more hydroxyl groups. The esters may also be fatty C6-30 carboxylic acid esters of glycerin or short chain C1-6 mono-, di-, or polyhydric alcohols such as ethylhexyl alcohol, or fatty alcohols having C6-30 alkyl chains such as cetyl, stearyl, isostearyl, behenyl, myristyl, alcohols; with fatty or aromatic carboxylic acids include myristic, stearic, behenic, isostearic, malic, adipic, sebacic, benzoic, lauric, caprylic, nonanoic, capric, linoleic, palmitic, linolenic acids and the like. Examples of such esters include but are not limited to ethylhexyl stearate; esters of glycerol or triglycerides, such as caprylic/capric triglycerides; glyceryl stearate; isononyl isononanoate; ethylhexyl isononanoate; diisostearyl malate; isostearyl malate; isostearyl palmitate; isostearyl isononanoate; and so on. Also suitable are oils such as cholesterol, and derivatives thereof.

Suitable hydrocarbons are particularly paraffinic hydrocarbons such as polybutene, polyisobutene, polydecene, or hydrogenated derivatives thereof.

Also suitable are certain types of fluorinated oils such as fluorinated silicones or ethers. Examples include trifluoropropyl polydimethylsiloxane; or perfluorinated ethers such as perfluoroisopropylether;

Humectants

The cosmetically acceptable carrier may also contain one or more humectants. If present, suggested ranges are from about 0.1-50%, preferably from about 0.5-45%, more preferably from about 1-40% by weight of the total composition. Suitable humectants include C1-6 alkylene glycols such as propylene, butylene, pentylene, hexylene glycols, etc. or polyhydric alcohols such as glycerin, or sugars such as sucrose, fructose, ribose, trehalose, galactose, lactose, and so one. Also suitable are humectants such as hyaluronic acid or neutralized derivatives thereof such as sodium hyaluronate.

Structuring Agents

The cosmetically acceptable composition may also comprise one or more structuring agents, that is, ingredients that increase the viscosity or otherwise thicken the composition. If present, suggested ranges are from about 0.01-75%, preferably from about 0.05-70%, more preferably from about 0.1-50% by weight of the total composition. Suitable structuring agents may be lipophilic or hydrophilic and may include waxes, fatty acids, and the like.

Examples of suitable waxes include animal, vegetable, mineral, silicone, or synthetic waxes such as polyethylene, candelilla, ozokerite, beeswax, synthetic wax, mimosa wax, bayberry, flower waxes, plant waxes. Suitable silicone waxes include alkyl or alkoxy dimethicones such as stearyl dimethicone, stearoxy dimethicone, behenyl dimethicone, behenoxydimethicone, and the like.

Also suitable as structuring agents are fatty acids that are solid or semi-solid at room temperature such as linoleic acid, stearic acid, and so on.

Also suitable as structuring agents are various types of saccharides and polysaccharides such as celluloses and alkyl celluloses, saccharides, saccharide isomerates, and so on.

Surfactants

The composition may also contain surfactants, including anionic, nonionic, amphoteric, zwitterionic, or betaine surfactants. If present, suggested ranges are from about 0.01-45%, preferably from about 0.05-40%, more preferably from about 0.1-35% by weight of the total composition. Particularly preferred are nonionic surfactants that are organic or silicone. Suitable nonionic silicone surfactants include those commonly referred to as dimethicone copolyol, cetyl dimethicone copolyol; or linear or crosslinked silicones substituted with ethylene oxide groups such as PEG-10 dimethicone, PEG, PPG, or combinations thereof of dimethicone crosspolymers such as PEG/PPG 10/1 dimethicone crosspolymer, PEG-10 lauryl dimethicone crosspolymer, PEG-15 lauryldimethicone crosspolymer, and the like.

Suitable organic nonionic surfactants include alkoxylated alcohols, particularly fatty C6-30 alkoxylated alcohols such as steareth, beheneth, ceteth, ceteareth, isosteareth, laureth, myristeth, where the number of repeating alkylene oxide units ranges from about 2 to 200; for example laureth-7, and so on.

Also suitable are alkylene oxides reacted with C6-30 fatty acids or fatty alcohols, as well as silicones containing alkylene oxides groups, such as PEG-substituted silicones, including PEG-(2-200) dimethicone where the number in the brackets indicates the number of repeating ethylene oxide groups.

Particulates

It may also be desirable to include particulates in the form of pigments, powders, or mixtures thereof in the composition. If present suggested ranges are from about 0.1-60%, preferably from about 0.5-50%, more preferably from about 1-45% by weight of the total composition. Suitable powders include titanium dioxide, boron nitride, mica, nylon, silica, talc, PMMA, acrylates copolymer, and the like.

It may also be desirable to include pigments or colorants in the composition. Suitable pigments include organic pigments such as D&C and FD&C colors or Lakes thereof, such as reds, yellows, greens, browns, etc. Also suitable are inorganic pigments such as iron oxides of yellow, red, black, or mixtures thereof.

Botanicals

It may also be desired to include one or more botanical extracts in the composition. If present, suggested ranges are from about 0.001 to 45%, preferably 0.005 to 40%, more preferably from about 0.01 to 35% by weight of the total composition. Examples of such extracts include those from grains, flowers, roots, seeds, stalks, peels, or leaves. Suitable grain extracts include those from wheat, barley, rice, corn, and the like. A wide variety of flower extracts may be used such as chamomile, poppy, rose, sunflower, pansy, and the like. Examples of extracts from seeds include grape seed, sunflower seeds, and so on. Examples of extracts from peels include orange, lemon, lime, mango, papaya, and the like. Leaf extracts may include aloe, spearmint, bayberry, etc.

Vitamins

Also suitable for use in the composition are various vitamins such as A, E, C, D, K, and their derivatives. Examples include retinol, retinyl palmitate, retinyl ascorbate, tocopherol, tocopheryl acetate, ascorbyl palmitate, and the like.

The composition of the invention may be in the form of a cream, lotion, solution, suspension, gel, mousse, spray. It may be liquid, solid, or semi-solid; anhydrous, aqueous based, or in the emulsion form—water in oil or oil in water emulsion.

In one form the composition may be in the emulsion form and contain the amounts of water and oil mentioned above, in addition to one or more of the other ingredients mentioned above.

The composition is applied to the affected areas of the body, which may be the skin on hips, buttocks or thighs. In the case where the composition is desired for sculpting facial or bodily surfaces, it is applied to the surface where it is desired to minimize the appearance of excess. For example, if a user desired to improve the appearance of a face or body areas that she perceived as being “too fat” she may wish to apply the composition of the invention to the areas of the face or body that she perceived as problematic. The composition may be applied once, twice, or more times per day. The applications may be in the morning, evening, or after exercise.

The invention will be further described in connection with the following examples which are set forth for the purposes of illustration only

### EXAMPLE 1

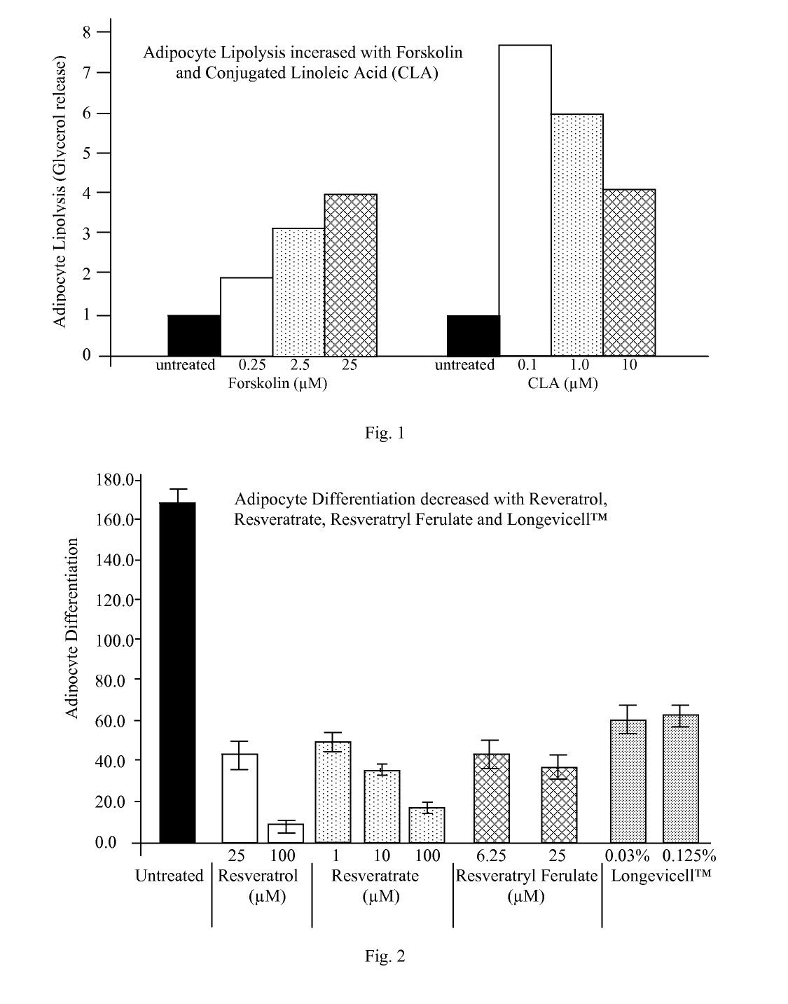

The adipocyte lipolytic activity of ingredients was tested using Lypolysis Assay Kit (dual glycerol and free fatty acids detection, reagents+cells) purchased from Zen-Bio (www.zen-bio.com) under catalog no. LIP-3. Serial dilutions of forskolin and conjugated linoleic acid were prepared in Krebs-Ringer Buffer containing 1% by weight free fatty acid bovine serum albumin. Culture medium was removed from the plate wells and the cells were washed with phosphate buffered saline. The forskolin test material was added to the test wells to provide wells having a total concentration of 25, 2.5, and 0.25 micromolar. The conjugated linoleic acid test material was added to the test wells to provide concentrations of 10, 1, and 0.1 micromolar. The plate was incubated for 5 hours at 37° C. Fifty microliters of test material was removed from each plate and transferred to clean wells. Triglyceride breakdown was tested by measuring for the presence of glycerol or NEFA (non-esterified fatty acids). To the wells being tested for glycerol, 50 microliters per well of the glycerol assay reagent from the kit was added and the plate was incubated at 25° C. for 15 minutes. To the wells being tested for NEFA, 50 microliters per well of NEFA solution A from the kit was added and the plate was incubated for 10 minutes at 37° C. Then 100 microliters per well of NEFA solution B was then added to the wells and the plate was incubated for another 10 minutes at 37° C. The plate with NEFA test wells was allowed to cool to room temperature for 5 minutes. The optical density of each well of the glycerol and NEFA plate wells was measured at 540 nanometers. The results are set forth in the table of FIG. 1.

It can be seen that both forskolin and conjugated linoleic acid were effective in increasing lipolysis of adipocytes.

### EXAMPLE 2

The adipocyte differentiation inhibition properties of various ingredients were tested using a lipid accumulation kit, Adipogenesis Assay Kit, PPARγ agonist (reagents+cells) having catalog no. DIF-AG purchased from Zen-Bio. Resveratrol, trisodium resveratrol triphosphate, resveratrol ferulate, and Longevicell™ purchased from Silab, which is an extract of myrtle. The test was conducted using the kit and following directions provided. Resveratrol at concentrations of 25 and 100 micromolar; trisodium resveratrol triphosphate at concentrations of 1, 10, and 100 micromolar; resveratrol ferulate at concentrations of 6.25 and 25 micromolar; and Longevicell at concentrations of 0.03% and 0.125% were tested. The results are set forth in the table of FIG. 2.

The above results demonstrate that all four test materials provide adipocyte differentiation inhibition activity.

### EXAMPLE 3

A cream for treating the body was prepared as follows:

| | % by |
|---|---|
| Ingredient | weight |
| Acrylamides copolymer | 0.03 |
| Saccharide isomerate | 1.00 |
| Caprylic/capric myristic/stearic triglyceride | 6.00 |
| Cholesterol | 0.20 |
| Mica/titanium dioxide | 0.75 |
| Fragrance | 0.75 |
| PEG-100 stearate | 1.30 |
| BHT | 0.09 |
| Dimethicone/polysilicone-11 | 4.00 |
| Trehalose | 0.001 |
| Ethylhexyl stearate | 1.70 |
| Phenoxyethanol | 0.03 |
| Caprylic/capric triglyceride | 4.50 |
| Squalane/hordeum vulgare (Barley) extract/ | 0.20 |
| triticum vulgare (wheat) germ extract | |
| Pentylene glycol | 1.50 |
| Polyperfluoromethylisopropyl ether | 0.3 |
| Linoleic acid | 0.20 |
| Glyceryl stearate | 1.50 |
| Butylene glycol | 0.02 |
| Tocopheryl acetate | 0.01 |
| PEG-10 dimethicone | 0.04 |
| Dihydroxyacetone | 1.50 |
| Citric acid | 0.6 |
| Aloe barbadensis leaf juice | 2.00 |
| Polyacrylamide/C13-14 isoparaffin/laureth-7 | 4.50 |
| Yellow #5 | 0.001 |
| Dimethicone | 1.07 |
| Isononyl isononanoate/ethylhexyl isononanoate | 0.30 |
| Sodium hyaluronate | 0.03 |
| Potassium sorbates | 0.10 |
| Glycerin | 3.00 |
| Trisodium resveratrol triphosphate | 1.00 |
| Conjugated linoleic acid | 0.50 |
| Water | QS |

The composition was prepared by combining the water phase ingredients separately and mixing well. The oil phase ingredients were combined. The water phase ingredients were dispersed into the oil phase with mixing to prepare an emulsion. The compositions were stored in jars.

What we claimed is:

1. A topical composition comprising at least one adipogenesis inhibitor and at least one adipocyte lipolytic agent and, optionally, at least one skin tightening agent.

2. The composition of claim 1 wherein the adipogenesis inhibitor and the adipocyte lipolytic agent are present in an amount sufficient to improve the appearance of the facial or bodily skin to which the composition is applied.

3. The composition of claim 1 which is in the form of an oil and water emulsion containing at least one silicone elastomer.

4. The composition of claim 3 additionally comprising at least one silicone surfactant.

5. The composition of claim 4 additionally comprising at least one botanical extract from grain.

6. The composition of claim 4 additionally comprising at least one ester of glycerin and a fatty C6-30 carboxylic acid.

7. The composition of claim 4 additionally comprising at least one PEG-dimethicone.

8. The composition of claim 1 wherein the adipogenesis inhibitor is resveratrol, an ester of resveratrol and an carboxylic acid, an ester of resveratrol and an inorganic acid, or mixtures thereof.

9. The composition of claim 1 wherein the adipocyte lipolytic agent comprises forskolin, conjugated linoleic acid, or mixtures thereof.

10. The composition of claim 1 wherein the adipogenesis inhibitor is selected from resveratrol, resveratrol ferulate, resveratrol triphosphate, trisodium resveratrol triphosphate, or mixtures thereof.

11. A method for treating the appearance of cellulite comprising topically applying to the affected area a composition comprising at least one adipogenesis inhibitor and at least one adipocyte lipolytic agent and, optionally, at least one skin tightening agent.

12. The method of claim 11 wherein the composition is applied in the form of a cream or lotion.

13. The method of claim 11 wherein the composition is applied at least twice per day.

14. A method for improving the appearance of facial or bodily skin surfaces or uneven skin surfaces comprising topically applying to the areas for which improvement is desired a composition comprising at least one adipogenesis inhibitor and at least one adipocyte lipolytic agent and, optionally, at least one skin tightening agent.

15. A method for treating the body to improve the appearance of dimpled skin caused by uneven fat deposits by applying a topical composition containing at least on adipogenesis inhibitor and at least one adipocyte lipolytic agent and, optionally, at least one skin tightening agent.
